(19) United States
(12) Patent Application Publication
(10) Pub. No.: US 2014/0235455 A1
(43) Pub. Date: Aug. 21, 2014

(54) USE OF HOXA9 GENE AS A BIOMARKER FOR THE DETECTION OF HEPATOCELLULAR CARCINOMA

(71) Applicants: Academia Sinica, Taipei (TW); Taipei Medical University, Taipei (TW)

(72) Inventors: Ching-Yu LIN, Taipei (TW); Jung-Chun LIN, Taipei (TW); Che-Chang CHANG, Taipei (TW); Yung-Kai HUANG, Taipei (TW); Guan Shuh BING, Taipei (TW); Hsiu-Wen HUANG, Taipei (TW); Ya-Wen LIN, Taipei (TW); Hung-Chung LAI, Taipei (TW); Yu-Lueng SHIH, Taipei (TW); Chung-Bao HSIEH, Taipei (TW); Chih-Chi KUO, Taipei (TW); Pei-Yu LIN, Taipei (TW); Ming-Song HSIEH, Taipei (TW); Chien-Jen CHEN, Taipei (TW)

(73) Assignees: ACADEMIA SINICA, Taipei (TW); TAIPEI MEDICAL UNIVERSITY, Taipei (TW)

(21) Appl. No.: 14/105,410

(22) Filed: Dec. 13, 2013

(30) Foreign Application Priority Data
Feb. 20, 2013 (TW) 102105786

(51) Int. Cl. C12Q 1/68

(52) U.S. Cl. CPC C12Q 1/6886
USPC 506/2; 435/6.11

(57) ABSTRACT

A method of detecting hepatocellular carcinoma includes the steps of: detecting a methylation level of a CpG site of HOXA9 gene in a biological sample taken from a suspected subject; and comparing the methylation level to a reference methylation level of a CpG site of HOXA9 gene in another biological sample taken from a normal subject not suffering from hepatocellular carcinoma, wherein when the methylation level is higher than the reference methylation level, the suspected subject is likely to suffer from hepatocellular carcinoma, and wherein each of the biological samples is selected from the group consisting of a blood sample, a serum sample, and a plasma sample.

### CROSS REFERENCE

The application claims priority from Taiwan Patent Application NO. 102105786, filed on Feb. 20, 2013, the content thereof is incorporated by reference herein.

### FIELD OF THE INVENTION

The invention is directed to use of HOXA9 gene as a biomarker for the detection of hepatocellular carcinoma. According to such use, the invention also provides a method of detecting hepatocellular carcinoma.

### BACKGROUND OF THE INVENTION

Hepatocellular carcinoma is one of the most common malignant tumors worldwide, and is difficult to be detected during the early stage, thus resulting in poor prognosis and high mortality. Because of the advancement of molecular biology, detailed understanding of molecular mechanisms involved in the development of hepatocellular carcinoma will help us to design better strategies for detection.

Recently, more evidence has reported that changes of DNA methylation patterns are highly associated with hepatocellular carcinoma. Especially, aberrant hypermethylation of tumor suppressor genes is not only observed in premalignant and malignant lesions of hepatocellular carcinoma, but also detected in serum/plasma of these concerned patients and their serum/plasma collected before clinic diagnosis, such as CDKN2A gene (Clin Cancer Res 2007; 138: 2378-2384). Therefore, it is believed that DNA methylation can be used for the detection of hepatocellular carcinoma.

The previous literature, J Clin Invest 2007; 1179: 2713-2722, has reported that tumor suppressor genes in hepatocellular carcinoma may be inactivated through methylation of their promoter regions. This implies that tumor suppressor genes may be potential methylation biomarkers for diagnosis and prognosis of hepatocellular carcinoma. However, most of the past studies were focused on the identification of a single gene or a few genes. Currently, methods for genome-wide methylation assay have been developed for the investigation of hepatocellular carcinoma. Furthermore, a new methodology using pooled DNA samples is also used to assess group DNA methylation average to reduce the amount of bisulfate-treated DNA for investigation.

### SUMMARY OF THE INVENTION

The invention is based on a study that a genome-wide methylation approach and a pooled DNA strategy are adopted for investigation of DNA methylation patterns in hepatocellular carcinoma, and then a potential methylation biomarker for hepatocellular carcinoma is identified. According to such study, it is proved that HomeoboxA9 (HOXA9) gene can be employed as a methylation biomarker for the detection of hepatocellular carcinoma.

One aspect of the invention is to disclose a method of detecting hepatocellular carcinoma, and the method includes the steps of: detecting a methylation level of a CpG site of HOXA9 gene in a biological sample taken from a suspected subject; and comparing the methylation level to a reference methylation level of a CpG site of HOXA9 gene in another biological sample taken from a normal subject not suffering from hepatocellular carcinoma, wherein when the methylation level is higher than the reference methylation level, the suspected subject is likely to suffer from hepatocellular carcinoma, and wherein each of the biological samples is selected from the group consisting of a blood sample, a serum sample, and a plasma sample.

In an embodiment of the invention, the detecting step is performed by using methylation-specific PCR (MS-PCR), quantitative methylation-specific PCR (Q-MSP), bisulfite sequencing (BS), microarray, mass spectrometer, denaturing high-performance liquid chromatography (DHPLC), pyrosequencing, or Southern blot assay to detect the methylation level.

In an embodiment of the invention, the CpG site of HOXA9 gene in the biological sample taken from the suspected subject includes a nucleotide sequence of SEQ ID NO: 1.

Another aspect of the invention is to disclose a method of detecting hepatocellular carcinoma, and the method includes the steps of: detecting a methylation level of a CpG site of HOXA9 gene and an expression level of α-fetoprotein in a biological sample taken from a suspected subject; and comparing the methylation level to a reference methylation level of a CpG site of HOXA9 gene in another biological sample taken from a normal subject not suffering from hepatocellular carcinoma, and determining the expression level, wherein when the methylation level is higher than the reference methylation level, and the expression level is higher than 10 ng/ml, the suspected subject is likely to suffer from hepatocellular carcinoma, and wherein each of the biological samples is selected from the group consisting of a blood sample, a serum sample, and a plasma sample.

In an embodiment of the invention, the detecting step is performed by using MS-PCR, Q-MSP, BS, microarray, mass spectrometer, DHPLC, pyrosequencing, or Southern blot assay to detect the methylation level.

In an embodiment of the invention, the detecting step is performed by using Western blot assay, enzyme-linked immunosorbent assay (ELISA), radioimmunoassay (RIA), or immunochromatographic test (ICT) to detect the expression level.

In an embodiment of the invention, the CpG site of HOXA9 gene in the biological sample taken from the suspected subject includes a nucleotide sequence of SEQ ID NO: 1.

### BRIEF DESCRIPTION OF THE DRAWINGS

FIG. 1 shows a flow chart illustrating the screening of methylation biomarkers for hepatocellular carcinoma.

FIG. 2(A) shows a schematic representation illustrating the promoter region of HOXA9 gene, in which each vertical line represents a CpG site.

FIG. 2(B) shows the reverse transcription PCR (RT-PCR) result of HOXA9 gene in different cell lines.

FIG. 2(C) shows the MS-PCR result of HOXA9 gene in different cell lines.

FIG. 2(D) shows the BS result of HOXA9 gene in different cell lines, in which each solid circle indicates a methylated CpG site, and each hollow circle indicates an unmethylated CpG site.

FIGS. 3(A) and 3(B) show the Q-MSP result of HOXA9 gene in different tissues.

FIG. 3(C) shows the receiver operating characteristic (ROC) curve of methylation of HOXA9 gene in hepatocellular carcinoma patients' tumorous tissues.

FIG. 4(A) shows the Q-MSP result of HOXA9 gene in plasma samples from different subjects.

FIG. 4(B) shows the ROC curve of methylation of HOXA9 gene in plasma samples from hepatocellular carcinoma patients.

FIG. 5 shows the methylation analysis of HOXA9 gene in hepatocellular carcinoma patients' tumorous tissues and their plasma samples.

### DETAILED DESCRIPTION OF THE INVENTION

The detailed description and preferred embodiment of the invention will be set forth in the following content, and provided for people skilled in the art so as to understand the characteristic of the invention.

### Experimental Material and Procedure

### I. Clinical Samples

For microarray, 5 normal liver hemangioma tissues, and 15 pairs of hepatocellular carcinoma patients' tumorous and peripheral non-tumorous tissues are obtained from Taiwan Liver Cancer Network. For validation, 29 normal liver hemangioma tissues are obtained from Taiwan Liver Cancer Network, and 40 pairs of hepatocellular carcinoma patients' tumorous and peripheral non-tumorous tissues, and plasma samples corresponding to these tissues are obtained from Tri-Service General Hospital. For another validation, 60 pairs of hepatocellular carcinoma patients' tumorous and peripheral non-tumorous tissues are obtained from Taiwan Liver Cancer Network. Clinicopathological characteristics of these patients concerned in validation are listed in Table 1. 34 control plasma samples are obtained from Shuang-Ho hospital. All of these clinical samples herein are approved by the Institutional Review Board of Taipei Medical University and the Taiwan Liver Cancer Network User Committee.

TABLE 1
Clinicopathological characteristics of patients

| | Number of cases | |
|---|---|---|
| Characteristics | Set 1 | Set 2 |
| Hepatocellular carcinoma patients | 40 | 60 |
| Age, mean ± SD | 60.3 ± 14.1 | 58.2 ± 15.2 |
| Gender | | |
| Male | 28 | 30 |
| Female | 12 | 30 |
| Viral infection | | |
| HBs-Ag-positive | 17 | 21 |
| Anti-HCV-positive | 12 | 20 |
| Double negative | 11 | 19 |
| Cirrhosis | | |
| No | 17 | 37 |
| Yes | 20 | 23 |
| Undetermined | 3 | 0 |
| α-fetoprotein expression level, | | |
| ng/ml | | |
| <10 | 10 | 19 |
| ≧10 | 28 | 41 |
| Undetermined | 2 | 0 |
| Tumor size, cm | | |
| <3 | 19 | 7 |
| ≧3 | 21 | 53 |
| Number of malignant nodules | | |
| Single | 19 | 42 |
| Multiple | 21 | 18 |
| TNM stage | | |
| Stage I, II | 26 | 41 |
| Stage III, IV | 13 | 19 |
| Undetermined | 1 | 0 |

### II. Cell Lines

A normal liver cell line, THLE-3, and hepatocellular carcinoma cell lines, HepG2, Hep3B, and SK-HEP1, are purchased from American Type Culture Collection. Other hepatocellular carcinoma cell lines, TONG, Mahlavu, PLC/PRF/5, HuH6, HuH7, and HA22T, are provided by Professor Kwang-Huei Lin (Chuang-Guan University, Taiwan). For the treatment of demethylating agent 5-aza-2′-deoxycytidine (5DAC), hepatocellular carcinoma cell lines are incubated according to the method described in Cancer 2006; 1073: 579-590.

### III. Methylation Array

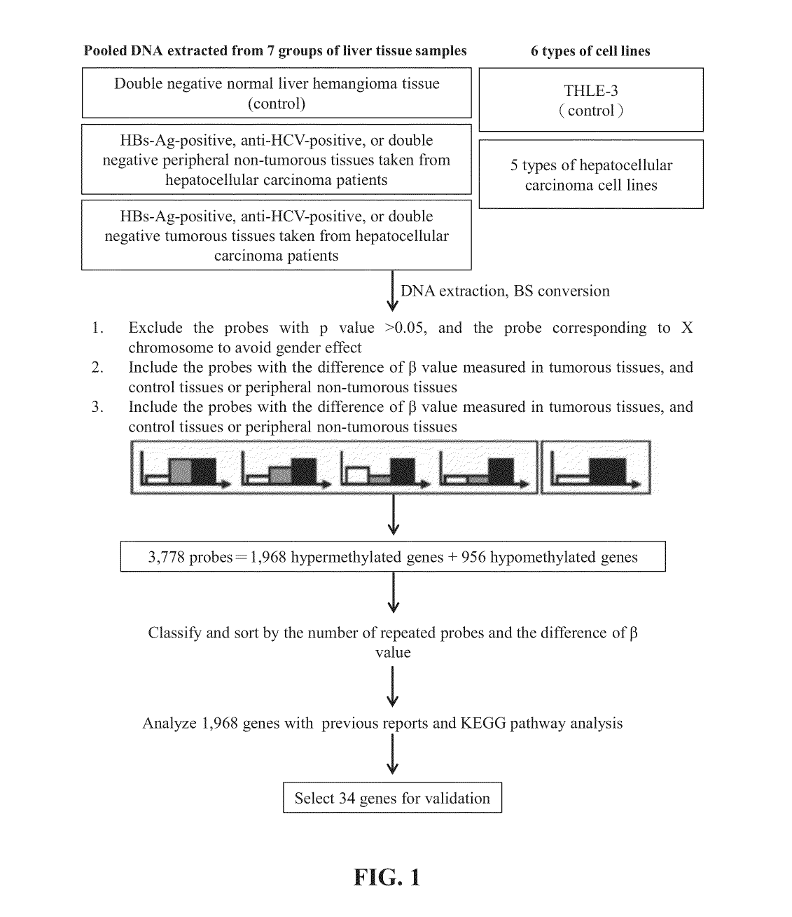

As shown in FIG. 1, liver tissues are divided into 7 groups, and pooled DNA extracted from each group and DNA extracted from each cell line are subject for Illumina Infinium Methylation Assay, where β value stands for methylation index outputted from each probe used in the assay. The value ranges from 0 to 1, and represents a ratio of the intensity of methylated signal with respect to the total signal. The value is used to calculate the difference of methylation level between these clinical samples.

### IV. Expression and Methylation Analysis

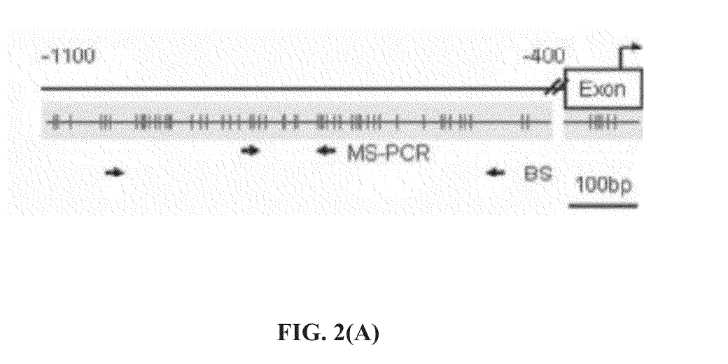

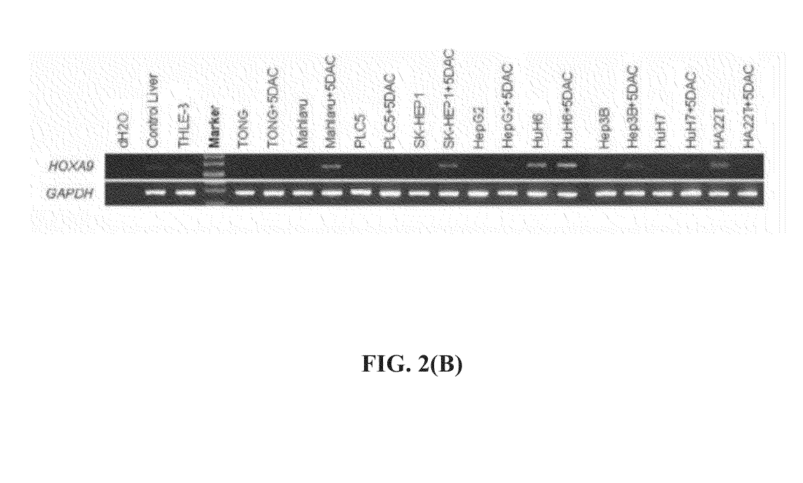

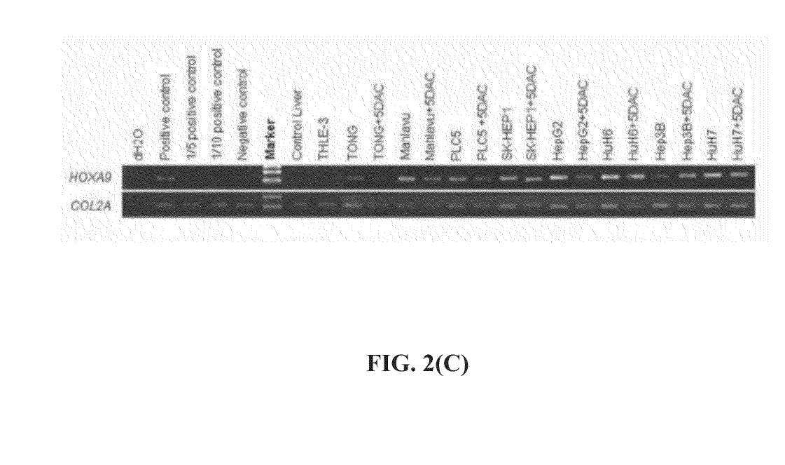

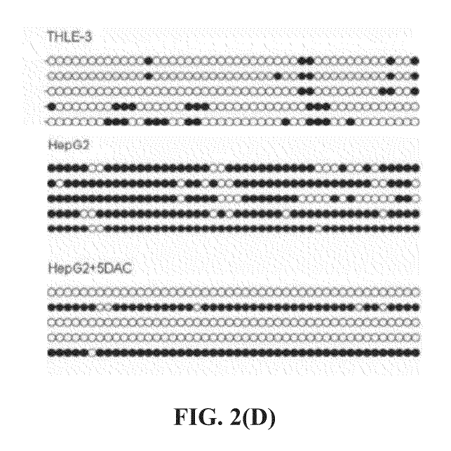

Expression and methylation analysis are performed according to the method described in Cancer 2006; 1073: 579-590 and Cancer 2010; 11618: 4266-4274. Nucleotide sequences of SEQ ID NO: 3 and SEQ ID NO: 4 are employed as MS-PCR primer pairs or Q-MSP primer pairs to detect the methylation level of HOXA9 gene; nucleotide sequences of SEQ ID NO: 5 and SEQ ID NO: 6 are employed as BS primer pairs to detect the methylation level of HOXA9 gene. As shown in FIG. 2(A), MS-PCR primer pairs or Q-MSP primer pairs correspond to a promoter region (SEQ ID NO: 1) of HOXA9 gene, and BS primer pairs correspond to another promoter region (SEQ ID NO: 2) of HOXA9 gene.

### Experimental Results

### I. Identification of Methylated Genes in Hepatocellular Carcinoma

As shown in FIG. 1, the methylation profiles of 27,578 CpG sites in liver tissues and cell lines are analyzed using a methylation array and a pooled DNA strategy. After the process of gene filtering, 3,778 probes (corresponding to 1,968 hypermethylated genes and 956 hypomethylated genes) are classified and sorted by the number of repeated probes and the difference of 13 value. Then, the 1,968 hypermethylated genes are analyzed with the previous reports including Carcinogenesis 2008; 2910: 1901-1910, Cancer Sci 2010; 1016; 1501-1510, PLoS One 2010; 53: e9749, J Korean Med Sci 2010; 258: 1152-1159, PLoS One 2011; 65: e19862, Int J Cancer 2012; 1306: 1319-1328, and Methods 2010; 523: 255-258, and ten pathways related to these hypermethylated genes are found using Kyoto Encyclopedia of Genes and Genomes (KEGG) pathway analysis (Table 2). Finally, 34 genes are selected for validation, and 16 of which are hypermethylated in hepatocellular carcinoma, and the others are identified in previous array data or in other cancers.

TABLE 2
Pathways related to hypermethylated genes

| Pathway | p value |
|---|---|
| Neuroactive ligand-receptor interaction | <0.0001 |
| Pathways in cancer | <0.0001 |
| Focal adhesion | <0.0001 |
| Calcium signaling pathway | <0.0001 |
| ECM-receptor interaction | 0.0004 |
| Wnt signaling pathway | 0.0036 |
| MAPK signaling pathway | 0.0082 |
| Axon guidance | 0.0115 |
| Leukocyte transendothelial migration | 0.0147 |
| Regulation of actin cytoskeleton | 0.0208 |

### II. Correlation of Expression of Selected Genes and Methylation of their Promoter Region in Cell Lines

Firstly, RT-PCR is performed to analyze the expression of selected genes in cell lines. The result is presented in FIG. 2(B). HOXA9 gene and other 28 genes are expressed in control liver tissues and THLE-3 cell line, with the exclusion of MSX1 gene, HOXD4 gene, CNTNAP2 gene, PLAU gene, and BMP gene. Furthermore, the 29 genes are downregulated in over half of the hepatocellular carcinoma cell lines. However, expression of part of the 29 genes are reinstated after the treatment of DNA methyltransferase inhibitor 5DAC, which interprets the downregulation of these genes are associated with DNA methylation.

Next, MS-PCR assay and BS assay are performed to analyze the methylation level of the 29 genes in cell lines. The MS-PCR result is presented in FIG. 2(C). Methylation of promoter regions of HOXA9 gene and other 24 genes are detected in hepatocellular carcinoma cell lines, with the exclusion of EYA4 gene, PYCARD gene, LOX gene, and F2R gene. Furthermore, after the treatment of 5DAC, the methylated bands are decreased. The BS result is presented in FIG. 2(D). The methylation level of promoter regions of HOXA9 gene in hepatocellular carcinoma cell lines is indeed higher than that in normal cell lines.

### III. Methylation Level of Selected Genes in Tissues

Firstly, MS-PCR is executed to determine the methylation frequency of HOXA9 gene and other 24 genes in tissues, and HOXA9 gene and 9 genes including NEUROG1, TNFESF10C, IRAK3, GFPT2, ZNF177, DPYSL4, ELOVL4, FSD1, and CACNA1G, are selected for further analysis in 29 normal liver hemangioma tissues and 30 pairs of hepatocellular carcinoma patients' tumorous tissues and peripheral non-tumorous tissues. Taken altogether, HOXA9 gene and the 9 selected genes are frequently methylated in tumorous tissues (Table 3, 33.3%-76.6%), and three methylation patterns are generalized from the 10 genes: (i) promoter regions of these genes are methylated in control liver tissues and tumorous tissues, but the methylation level in tumorous tissues is higher than that in control liver tissues, such as HOXA9 gene; (ii) promoter regions of these genes are methylated in control liver tissues and tumorous tissues, such as NEUROG1 gene; (iii) promoter regions of these genes are only methylated in tumorous tissues, such as ZNF177 gene. Among the 10 genes, HOXA9 gene is frequently methylated in tumorous tissues (76.7%, 23/30), but rarely in control liver tissues (8.3%, 2/24). Therefore, HOXA9 gene is relatively significant.

TABLE 3
Methylation frequency of 10 selected genes in liver tissues

| | Number of methylated tissues(%) | | | | | | |
|---|---|---|---|---|---|---|---|
| | Control | | Hepatocellular carcinoma | | | | |
| | liver | Hepatitis | patients' non-tumorous tissues | | | Tumorous | |
| | tissues, | tissues, | —, | Hepatitis, | Cirrhosis, | tissues, | |
| Symbol | n = 16 | n = 13 | n = 8 | n = 9 | n = 13 | n = 30 | p value |
| HOXA9 | 0(0.0%) | 1(7.7%) | 2(25.0%) | 4(44.4%) | 10(76.9%) | 23(76.7%) | <0.0001 |
| NEUROG1 | 6(37.5%) | 7(53.8%) | 4(50.0%) | 7(77.8%) | 11(84.6%) | 23(76.7%) | 0.0021 |
| TNFRSF10C | 5(31.3%) | 4(30.8%) | 1(12.5%) | 4(44.4%) | 4(30.8%) | 20(66.7%) | 0.0086 |
| IRAK3 | 0(0.0%) | 0(0.0%) | 1(12.5%) | 0(0.0%) | 1(7.7%) | 12(40.0%) | 0.0001 |
| GFPT2 | 0(0.0%) | 1(7.7%) | 0(0.0%) | 2(22.2%) | 1(7.7%) | 13(43.3%) | 0.0002 |
| ZNF177 | 0(0.0%) | 0(0.0%) | 0(0.0%) | 0(0.0%) | 0(0.0%) | 19(63.3%) | <0.0001 |
| DPYSL4 | 4(25.0%) | 5(38.5%) | 0(0.0%) | 3(33.3%) | 7(53.8%) | 21(70.0%) | 0.0007 |
| ELOVL4 | 0(0.0%) | 1(7.7%) | 0(0.0%) | 1(11.1%) | 4(30.8%) | 2(66.7%) | <0.0001 |
| FSD1 | 1(6.3%) | 1(7.7%) | 0(0.0%) | 1(11.1%) | 0(0.0%) | 13(43.3%) | 0.0013 |
| CACNA1G | 0(0.0%) | 0(0.0%) | 0(0.0%) | 1(11.1%) | 1(7.7%) | 10(33.3%) | 0.0004 |

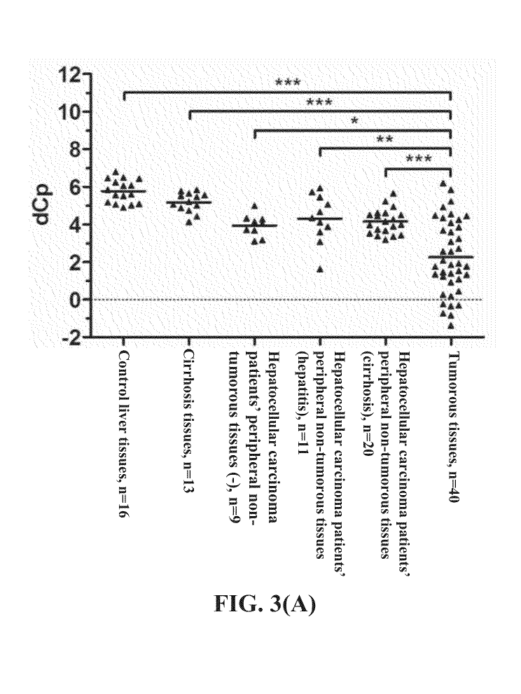

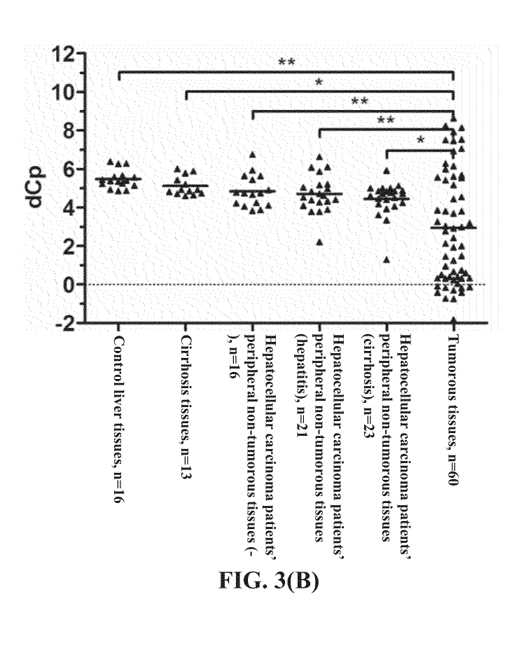

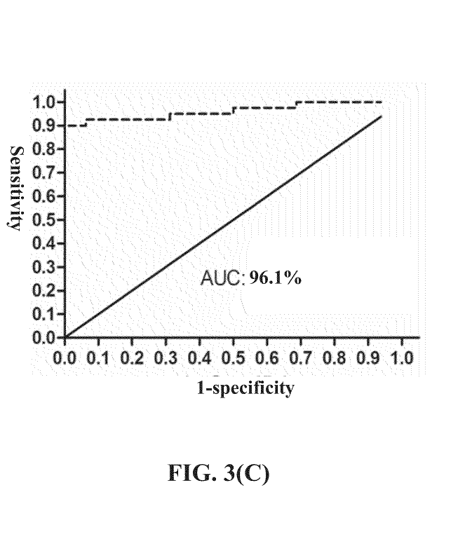

After that, BS and Q-MSP are executed to further determine the methylation level of HOXA9 gene in different tissues. The Q-MSP result is presented in FIGS. 3(A) and 3(B). The methylation level described herein is expressed as dCp value, and this value is a difference of the cycle thread value of HOXA gene measured in Q-MSP minus that of internal control gene, COL2A. Generally, dCp value described in the following content complies with the definition previously defined. As shown in FIGS. 3(A) and 3(B), dCp value of HOXA gene in tumorous tissues is lower than that in other tissues, which indicates that the methylation level of HOXA9 gene in hepatocellular carcinoma patients' tumorous tissues is relatively high. Area under the ROC curve (AUC) of methylation of HOXA9 gene is employed to distinguish tumorous tissues and control tissues, and AUC is approximately of 0.961 (FIG. 3(C)).

As listed in Table 4, HOXA9 gene has sensitivity for the detection of hepatocellular carcinoma higher than that of CDKN2A gene, a well-known biomarker for hepatocellular carcinoma (92.5% vs. 77.5%), and identical specificity for the detection of hepatocellular carcinoma with that of CDKN2A gene, so that HOXA9 gene is preferably used for the detection of hepatocellular carcinoma. Sensitivity and specificity for the detection of hepatocellular carcinoma are of 95.0% and 87.5% in combined test of HOXA9 gene and CDKN2A gene, respectively. However, from Table 5, there is no correlation between methylation of HOXA9 gene, methylation of CDKN2A gene, and clinicopathological characteristics of these concerned patients.

TABLE 4
Sensitivity and specificity for detection of hepatocellular carcinoma
using methylation level HOXA9 gene and CDKN2A gene

| | Best cut-off | | |
|---|---|---|---|
| Symbol | value | Sensitivity | Specificity |
| HOXA9 | dCp < 4.97 | 92.5% | 93.8% |
| CDKN2A | dCp < 9.04 | 77.5% | 93.8% |
| HOXA9 or | | 95.0% | 87.5% |
| CDKN2A | | | |
| HOXA9 and | | 75.0% | 100.0% |
| CDKN2A | | | |

TABLE 5
Correlation between methylation of HOXA9 gene,
methylation of CDKN2A gene, and clinicopathological
characteristics of these concerned patients

| | HOXA9, dCp | | Case | p |
|---|---|---|---|---|
| Characteristics | ≧4.97 | <4.97 | number | value |
| CDKN2A, dCp | | | | |
| ≧9.04 | 2 | 7 | 40 | 0.1215 |
| <9.04 | 1 | 30 | | |
| Age | | | | |
| <60 | 2 | 17 | 40 | 0.5962 |
| ≧60 | 1 | 20 | | |
| Gender | | | | |
| Female | 2 | 10 | 40 | 0.2093 |
| Male | 1 | 27 | | |
| Viral infection | | | | |
| HBs-Ag-positive | 2 | 15 | 40 | 0.6883 |
| HCs-Ag-positive | 0 | 12 | | |
| Double negative | 1 | 10 | | |
| Cirrhosis | | | | |
| No | 2 | 15 | 37 | 0.5843 |
| Yes | 1 | 19 | | |
| α-fetoprotein expression level, | | | | |
| ng/ml | | | | |
| <10 | 1 | 9 | 38 | 1.0000 |
| ≧10 | 2 | 26 | | |
| Tumor size, cm | | | | |
| <3 | 1 | 18 | 40 | 1.0000 |
| ≧3 | 2 | 19 | | |
| Number of malignant nodules | | | | |
| Single | 1 | 18 | 40 | 1.0000 |
| Multiple | 2 | 19 | | |
| TNM Stage | | | | |
| Stage I, II | 1 | 25 | 39 | 0.2532 |
| Stage III, IV | 2 | 11 | | |

From the above experimental result, it is demonstrated that HOXA gene and the selected genes have a higher methylation level in hepatocellular carcinoma patients' tumorous tissues than that of the same gene in other tissues.

### IV. Methylation Level of HOXA9 Gene in Serum Samples Corresponding to Hepatocellular Carcinoma Patients' Tumorous Tissues

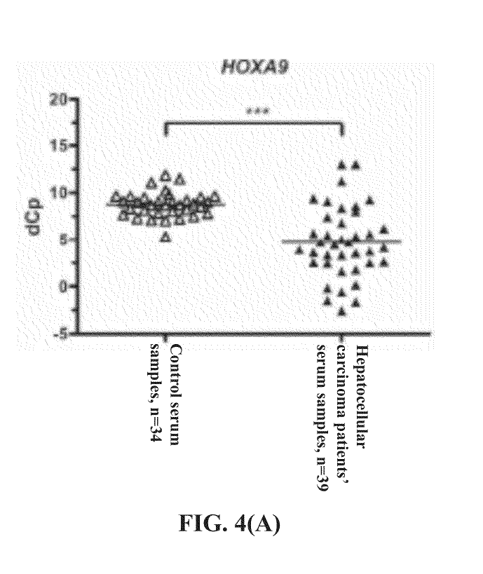

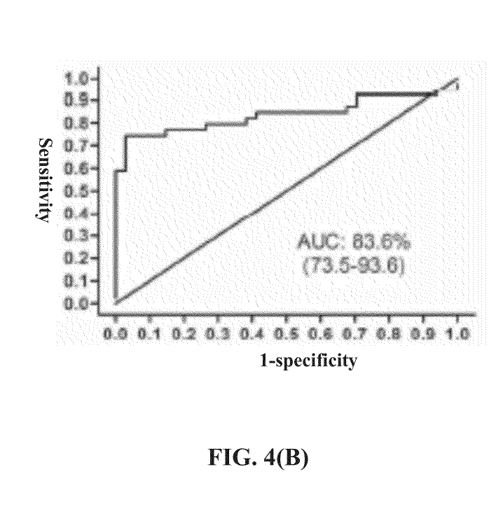

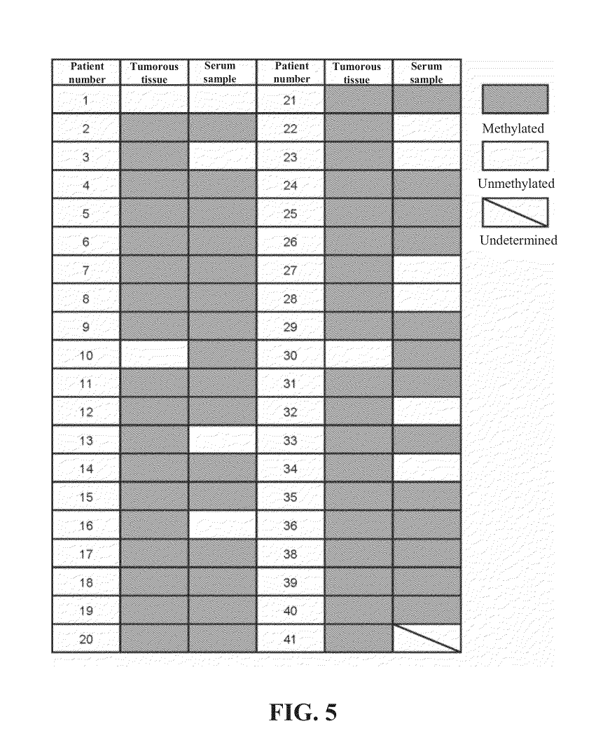

Q-MSP is performed to distinguish the methylation level of HOXA9 gene in control serum samples and that in hepatocellular carcinoma patients' serum samples. The methylation level of HOXA9 gene in hepatocellular carcinoma patients' serum samples is higher than the methylation level of HOXA9 gene in control serum samples (FIG. 4(A)). Additionally, the methylation of CDKN2A gene can't be detected in hepatocellular carcinoma patients' serum samples and control serum samples. AUC of methylation of HOXA9 gene is employed to distinguish hepatocellular carcinoma patients' serum samples and control serum samples, and AUC is approximately of 0.836 (FIG. 4(B)).

As listed in Table 6, sensitivity and specificity of HOXA9 gene for the detection of hepatocellular carcinoma are of 73.0% and 97.1%, respectively. Sensitivity for the detection of hepatocellular carcinoma is of 97.1% in combined test of HOXA9 gene and α-fetoprotein.

TABLE 6
Sensitivity and specificity for detection of hepatocellular carcinoma
using methylation level HOXA9 gene and expression level
of α-fetoprotein in hepatocellular carcinoma patients' serum
samples and control serum samples

| Symbol | Best cut-off value | Sensitivity | Specificity |
|---|---|---|---|
| HOXA9 | dCp < 6.93 | 73.0% | 97.1% |
| AFP | >10 ng/ml | 73.0% | 100.0% |
| HOXA9 or AFP | | 94.6% | 97.1% |
| HOXA9 and AFP | | 51.4% | 100.0% |

40 hepatocellular carcinoma patients' tumorous tissues and their serum samples are analyzed, and 29 of these serum samples exhibit methylation of HOXA9 gene.

As stated above, it has been proved that a methylation level of a CpG site of HOXA9 gene in a serum sample, a plasma sample or a blood sample taken from a suspected subject can be used to determine whether the suspected subject is likely to suffer from hepatocellular carcinoma. Besides, the sample is easily obtained using medical techniques, which brings both convenience and accuracy for the detection of hepatocellular carcinoma.

While the invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A method of detecting hepatocellular carcinoma, comprising:
detecting a methylation level of a CpG site of HOXA9 gene in a biological sample taken from a suspected subject; and
comparing the methylation level to a reference methylation level of a CpG site of HOXA9 gene in another biological sample taken from a normal subject not suffering from hepatocellular carcinoma;
wherein when the methylation level is higher than the reference methylation level, the suspected subject is likely to suffer from hepatocellular carcinoma; and
wherein each of the biological samples is selected from a group consisting of a blood sample, a serum sample, and a plasma sample.

2. The method as claimed in claim 1, wherein each of the biological samples is a serum sample.

3. The method as claimed in claim 1, wherein the detecting step is performed by using methylation-specific PCR, quantitative methylation-specific PCR, bisulfite sequencing, microarray, mass spectrometer, denaturing high-performance liquid chromatography, pyrosequencing, or Southern blot assay to detect the methylation level.

4. The method as claimed in claim 1, wherein the CpG site of HOXA9 gene in the biological sample taken from the suspected subject includes a nucleotide sequence of SEQ NO. 1.

5. The method as claimed in claim 2, wherein the CpG site of HOXA9 gene in the biological sample taken from the suspected subject includes a nucleotide sequence of SEQ NO. 1.

6. A method of detecting hepatocellular carcinoma, comprising:
detecting a methylation level of a CpG site of HOXA9 gene and an expression level of α-fetoprotein in a biological sample taken from a suspected subject; and
comparing the methylation level to a reference methylation level of a CpG site of HOXA9 gene in another biological sample taken from a normal subject not suffering from hepatocellular carcinoma, and determining the expression level;
wherein when the methylation level is higher than the reference methylation level, and the expression level is higher than 10 ng/ml, the suspected subject is likely to suffer from hepatocellular carcinoma; and
wherein each of the biological samples is selected from a group consisting of a blood sample, a serum sample, and a plasma sample.

7. The method as claimed in claim 6, wherein each of the biological samples is a serum sample.

8. The method as claimed in claim 6, wherein the detecting step is performed by using methylation-specific PCR, quantitative methylation-specific PCR, bisulfite sequencing, microarray, mass spectrometer, denaturing high-performance liquid chromatography, pyrosequencing, or Southern blot assay to detect the methylation level.

9. The method as claimed in claim 6, wherein the CpG site of HOXA9 gene in the biological sample taken from the suspected subject includes a nucleotide sequence of SEQ NO. 1.

10. The method as claimed in claim 7, wherein the CpG site of HOXA9 gene in the biological sample taken from the suspected subject includes a nucleotide sequence of SEQ NO. 1.

11. The method as claimed in claim 6, wherein the detecting step is performed by using Western blot assay, enzyme-linked immunosorbent assay, radioimmunoassay, or immunochromatographic test to detect the expression level.


SEQUENCE LISTING (6 sequences)

SEQ ID NO: 1 - DNA, 532 bp, Homo sapiens
  Promoter region of HOXA9 gene
gcctgggaatcctgattgccagctgatgagaaaggcgggctgggcgcgcgtgtgcgtggggtcgagggccggggaccgagcgcgccgcacaaccaaccaggccctcaaaaccttcgccctggtggcggctggccgctccctcctggccagctcctccgtggggtcctcgtagcaaaggcgaatttaagggttgcccgggcgcccctcgctccaggcgggtagctgtggggacctacacccgcggtactccctgagcggccggtccctgcctggagtgccctggtagggccggcggcggctccgtttgggacggatcctgcgttgaatttgacttttcgagggcggccgcgggtaaactcgcctctcccggggaccgcagggattatttacagggagctcgccaaccaaacacaacagtctaacctttccaagtcctcgtaaatttttacagctgggagccacggcgaggcaaacgaatctgttggtcgtttccgacttcccgccagcctgtgtggcttctgaaacaataact

SEQ ID NO: 2 - DNA, 122 bp, Homo sapiens
  Promoter region of HOXA9 gene
cgggcgcccctcgctccaggcgggtagctgtggggacctacacccgcggtactccctgagcggccggtccctgcctggagtgccctggtagggccggcggcggctccgtttgggacggatcc

SEQ ID NO: 3 - DNA, 28 bp, Artificial
  MS-PCR forward primer or Q-MSP forward primer
gcctgggaatcctgattgccagctgatg

SEQ ID NO: 4 - DNA, 28 bp, Artificial
  MS-PCR reverse primer or Q-MSP reverse primer
agttattgtttcagaagccacacaggct

SEQ ID NO: 5 - DNA, 21 bp, Artificial
  OTHER INFORMATION BS-PCR forward primer
cgggcgcccctcgctccaggc

SEQ ID NO: 6 - DNA, 22 bp, Artificial
  BS-PCR reverse primer
ggatccgtcccaaacggagccg